(19) United States
(12) Patent Application Publication
(10) Pub. No.: US 2025/0161630 A1
(43) Pub. Date: May 22, 2025

(54) Versatile Delivery Catheter

(71) Applicant: Unity Medical Inc., Edina, MN (US)

(72) Inventor: Matthew F. Ogle, Edina, MN (US)

(73) Assignee: Unity Medical Inc., Edina, MN (US)

(21) Appl. No.: 18/955,284

(22) Filed: Nov. 21, 2024

Related U.S. Application Data
(60) Provisional application No. 63/602,310, filed on Nov. 22, 2023.

(51) Int. Cl. A61M 25/00

(52) U.S. Cl. CPC A61M 25/0097

(57) ABSTRACT

A system for adjusting instrumentation during a vascular interventional procedure includes a flexible shaft having a lumen for passing a flexible extending element therethrough, and a removable instrument hub releasably attachable to the flexible shaft. The hub may include a hub element with grasping feature(s) and an opening for passing the extending element therethrough, and a tubular member extending from a distal end of the hub element. The system may include mating feature(s) arranged on the proximal end of the flexible shaft and/or a distal end of the tubular member, the mating feature(s) configured to frictionally mate with an interior surface of the other of the flexible shaft or tubular member, such that, upon insertion of the mating feature(s), the hub is releasably coupled to the flexible shaft and the tubular member is in fluid communication with the flexible shaft.

### RELATED APPLICATIONS

The present application claims priority to U.S. Provisional Patent Application Ser. No. 63/602,310 entitled “Versatile Delivery Catheter” and filed Nov. 22, 2023. All above-noted applications are hereby incorporated by reference in their entireties.

### BACKGROUND

Catheters designed for interventional diagnosis and treatment of vascular disease have been utilized for more than 30 years. Catheters serve as facilitating devices in interventional medical procedures, requiring a delicate balance of rigidity for navigating the vasculature effectively and flexibility to maneuver through intricate and winding blood vessel pathways. These catheters also play a dual role, delivering therapies and offering convenience to the distal part of the artery, which can involve tasks such as imaging, aspiration, or delivering other medical components such as wires or probes.

Guidewires, equally crucial in interventional procedures, are specifically designed for navigation, visualization, and facilitating the delivery of catheters to access and maintain distal vascular access. In the context of procedures in the brain's blood vessels, which are becoming increasingly vital for addressing acute stroke events and other interventions, the tortuous paths of these vessels pose a significant challenge in reaching target locations. Likewise, many other vessels within the body also feature winding pathways, intensifying the complexity of accessing specific targets.

Guidewires are routinely employed to establish access within a vessel, whether it's a blood vessel or another vascular structure. Typically, the guidewire is positioned at an early stage of a procedure, and then additional intervention devices can be guided over the guidewire to reach the intended target location. Following the placement of these devices, the guidewire may or may not be removed for the remainder of the procedure, making it a vital tool for catheter exchange.

Given the intricate nature of blood vessels, particularly in the brain, it becomes advantageous to have an intravascular tool that combines the best attributes of both a guidewire for exchange and a catheter for therapy. Such a versatile tool could significantly enhance the efficiency and precision of interventional procedures, especially in complex vascular scenarios.

Aneurysm treatment, often referred to as “Coiling,” involves repairing defects in arteries, such as aneurysms, by inserting a coil to prevent further complications.

Angioplasty is a medical intervention where a catheter with an inflatable balloon is used to open narrowed or obstructed arteries or veins, improving blood flow.

Stenting is a procedure in which a stent is deployed within an artery to address narrowing or blockages, maintaining a clear passage for blood flow.

In neurovascular stroke therapy (thrombectomy), a catheter is employed to remove obstructions with aspiration or other mechanical means within the arteries, thereby restoring blood flow. This is a critical procedure for addressing stroke-related conditions.

Ischemic strokes are often caused by clots within cerebral arteries, leading to blocked blood flow and deprivation of blood supply to brain tissues. Reducing the impact of this blood flow interruption is time-sensitive, with a priority on restoring blood flow as quickly as possible. The cerebral artery system is complex, highly branched, and circuitous, requiring medical treatment devices specially designed to navigate this intricate network for optimal placement.

Advances in interventional technology have introduced delivery of catheters as an initial step in certain procedures, such as those identified above. Despite these innovations, some existing devices lack the combination of desirable features.

Due to the limitations of current devices in the marketplace, the inventor recognized a need for alternative solutions that address the above challenges. The inventor addition recognized a need for any new catheter designs to enhance the efficiency and safety of catheter-based medical procedures.

### SUMMARY OF ILLUSTRATIVE EMBODIMENTS

The present disclosure relates to improved methods and instrumentation for performing vascular interventional procedures involving the use of catheters. In some embodiments, the methods and instrumentation focus on the effective delivery of various catheters to distal vascular anatomy while ensuring that vascular access remains intact and minimizing the risk of additional complications. Various methods and instrumentation described herein are applicable to a range of complex interventional medical procedures, including, in some examples, neurovascular stroke therapy (thrombectomy), aneurysm, angioplasty, and stenting.

A key disadvantage with many of the forementioned complex interventional procedures is that a variety of specialized catheters have been created using customized intervention designs. While the existing catheter designs may be well-suited for a specific purpose, in some instances, medical personnel will discover that the initially deployed catheter is not suitable for the ongoing procedure. This, in turn, can create timing and/or safety concerns in exchanging instrumentation or otherwise trying to work with ill adapted instrumentation for the present medical condition.

One challenge lies in the hub which is located at the proximal end of the catheter and complicates catheter removal. Frequently, the hub is built-in or otherwise not readily detachable to allow for catheter removal without removing the guidewire that has already been positioned at the procedure site. Even where the hub may be detached, removal of the catheter can result in a “free floating” guide wire that migrates from the desired position during catheter removal.

In one aspect, the present disclosure relates to methods and instrumentation for securing the position of a flexible extending element (e.g., guidewire, microcatheter, etc.) while exchanging outer instrumentation (e.g., an outer delivery element) such as one catheter for another catheter. In some embodiments, the position of the flexible extending element is secured by removable or releasable deployment of an external securing system designed to prevent forward and backward movement of the guidewire, thereby effectively locking the present position of the guidewire. The external securing system (e.g., microcatheter, metal tubing (“hypotube”), sleeve, etc.) may also aid in preventing blood loss during the procedure. In some embodiments, a shape of the flexible extending element is modified to enable or prevent movement. In one example, a curved (e.g., “S”-shaped) portion of the flexible extending element may be straightened by an external support element (e.g., microcatheter, hypotube, sleeve, etc.), thereby enabling forward and backward movement of the flexible extending element. In another example, a curved (e.g., “S”-shaped) external support element (e.g., microcatheter, hypotube, sleeve, etc.) may be slid upon a portion of the flexible extending element, thereby frictionally retaining the flexible extending element within the curved portion.

In some embodiments, a wedge element is disposed over the flexible extending element, such that the wedge blocks backward migration of the flexible extending element upon bumping against an outer delivery element (e.g., microcatheter or catheter). The wedge element, for example, may be formed to snugly fit between a guidewire and a microcatheter, such that the wedge element may be slid between the microcatheter and the guidewire to hold the guidewire in place.

In some embodiments, a stretchable retention sleeve is provided surrounding at least a portion of the length of the flexible extending element. The retention sleeve, for example, may mechanically retain the position of the flexible extending element via compressive force against the retention sleeve. In operation, the retention sleeve may be stretched over the proximal end of the flexible shaft such that the retention sleeve is applying compressive force to both the proximal end of the flexible shaft and an exposed section of the flexible extending element proximal to the proximal end of the flexible shaft. The retention sleeve, for example, may be sufficiently thin to allow the advancement of additional sheaths, shafts, and/or catheters over the outer diameter of the flexible shaft, ensuring a seamless exchange or movement of medical devices within the vasculature.

In some embodiments, to facilitate this secure attachment, the flexible shaft is designed with differential diameters, such that the proximal end of the flexible shaft where the sleeve element would be pulled over is smaller in diameter than a region distal to this section. The multi-diameter design, for example, will enable effective accommodation of the sleeve element while ensuring additional external instrumentation (catheter, sheath, etc.) can be maneuvered through tortuous vascular pathways with precision and minimal risk. Despite multiple external diameters, an interior diameter of the flexible shaft, in some implementations, is consistent. In illustration, the interior diameter may be 0.019″, while the exterior diameter varies from 0.062″ to 0.082.”

After securing the position of the flexible extending element, in some embodiments, exchange of delivery elements (e.g., catheters) is carried out, potentially replacing a present catheter with a more appropriate catheter for the task at hand. To enable exchange, in some implementations, a hub end of the delivery element is replaceably removed.

In one aspect, the present disclosure relates to an instrumentation system including a removal hub system configured to engage with the proximal end of a delivery element (e.g., catheter) having a hollow (e.g., tubular) or multi-lumen shaft through which a flexible extending element can be guided to a procedure site via a tortuous vascular path. The removable hub system, in some embodiments, includes a shaft mating element for releasably connecting to each of the flexible shaft element and a hub element. The shaft mating element may include or be surrounded by a two-piece locking shaft element configured to close around the proximal end of the delivery element. The pieces of the two-piece locking shaft element, for example, may be hinged on one edge and include mating lock elements on another edge to releasably close around the circumference of the proximal end of the catheter. The proximal end of the catheter, for example, may include a larger diameter end section, and the two-piece locking shaft element may narrow at its distal end such that the end section is captured by the two-piece locking shaft element. The larger diameter end section, for example, may enable ease of manipulation when locking the hub element to the flexible shaft.

The removable hub system, in some embodiments, includes a locking shaft element configured to releasably surround the circumference of the proximal end of the catheter and having at least one connector element for releasably connecting an instrument hub. The instrument hub, for example, may be configured to receive a guidewire, a fluid or other therapeutic substance, and/or additional instrumentation. The locking shaft element, for example, may include a threaded end for releasably engaging the complementary threaded end of the instrumentation hub. In another example, the locking shaft element may include a first portion of a luer lock connector configured to engage an instrumentation hub having the second portion of the luer lock connector.

In some embodiments, a removable hub system includes a tubular extension for mating with a delivery element (e.g., catheter). The tubular extension may be formed of rigid or semi-rigid material. The tubular extension, for example, may be formed of a metal tubing, such as a hypotube. In another example, the tubular extension may be formed of one or more polymers.

The tubular extension, in some embodiments, includes one or more mating features for frictionally mating with an interior surface of a lumen of the delivery element. The mating feature(s), in some implementations, are formed of a rigid material similar in greatest diameter to an interior diameter of the proximal end of the delivery element, such that the delivery element stretches to snugly fit over the mating feature(s). In some implementations, the mating feature(s) are formed of a flexible material designed to displace, deform, and/or compress when inserted into the lumen of the delivery element such that, after insertion, the mating feature(s) apply a frictional force, thereby retaining the position of an inserted section of the tubular extension in a proximal end of the delivery element. The mating feature(s), in some examples, may include one or more barbs, ramp or wedge surfaces, ledges, wings, bumps, and/or protrusions. The mating feature(s) may be arranged to contact the interior surface of the delivery element lumen over a length of the delivery element of at least one centimeter. The mating feature(s) may be arranged to contact at least one fifth of the circumference of the interior surface.

The mating feature(s), in some implementations, are arranged on a protrusion extending from the distal end of the tubular extension. The protrusion may be formed from or anchored to an interior surface of the tubular extension. The mating feature(s) may be integral with or mounted to the protrusion. The protrusion and the mating feature(s) may be designed to allow for fluid communication between the removable hub system and the delivery element.

In some implementations, a proximal end of the delivery element includes more mating feature(s) for frictionally mating with an interior surface of the tubular extension of the removable hub system. The mating feature(s), in some implementations, are formed of a rigid material similar in greatest diameter to an interior diameter of the distal end of the tubular extension of the removable hub system, such that the tubular extension stretches to snugly fit over the mating feature(s). In some implementations, the mating feature(s) are formed of a flexible material designed to displace, deform, and/or compress when inserted into the tubular extension of the removable hub system such that, after insertion, the mating feature(s) apply a frictional force, thereby retaining the position of an inserted section of the proximal end of the delivery element in the distal end of the tubular extension. The mating feature(s), in some examples, may include one or more barbs, ramp or wedge surfaces, ledges, wings, bumps, and/or protrusions. The mating feature(s) may be arranged to contact the interior surface of the tubular extension over a length of at least one centimeter. The mating feature(s) may be arranged to contact at least one fifth of the circumference of the interior surface of the tubular extension.

In some implementations, the mating feature(s) of the proximal end of the delivery element are arranged on a protrusion extending from the proximal end of the delivery element. The protrusion may be formed from or anchored to an interior surface of a lumen of the delivery element. The mating feature(s) may be integral with or mounted to the protrusion. The protrusion and the mating feature(s) may be designed to allow for fluid communication between the removable hub system and the delivery element.

In some implementations, a distal end of the tubular extension of the removable hub element includes one or more mating features configured to frictionally and/or lockably mate with one or more corresponding mating features of a proximal end of the delivery element. The mating features, in a first example, may include one or more protrusions configured to mate with one or more detents, such as a ridge feature of one of the tubular extension or the delivery element designed to mate with a valley feature of the other of the tubular extension or the delivery element. In a second example, the mating features may include a set of wedges or teeth within the lumen of the tubular extension designed to frictionally or lockably interlace with corresponding wedges or teeth within the lumen of the delivery element. In a third example, the mating features may include a threaded connection configured to lock the tubular extension with the delivery element using a twisting motion.

In some implementations, an interior lumen of the tubular extension includes a flared diameter, such that the proximal end of the delivery element may be inserted into the end of the tubular extension of the removable hub system and pushed into the “funnel” shape of the interior surface of the tubular extension to a point where the proximal end of the delivery element snugly fits within the interior diameter of the tubular extension. In this manner, the ends may be more easily aligned, providing the benefit of swift connection in a surgical setting. The interior surface of the flared lumen of the tubular extension may include a sticky, tacky, or textured surface finish designed to frictionally retrain the proximal end of the delivery element.

In one aspect, the present disclosure relates to methods and systems for selectively extending the length of a delivery element, such as a catheter. A primary delivery element (e.g., designed for manipulation by a medical professional outside of the body of the patient), for example, may be designed to interface with an extension delivery element to frictionally or lockably mate the primary delivery element with the extension delivery element. The connection mechanisms used to connect the primary delivery element with the extension delivery element may include any of the above examples presented in relation to mating the tubular extension with the delivery element. The primary delivery element, for example, may be configured to interface at its proximal end with a removable hub system. The extension delivery element, in a further example, may be designed for increased flexibility and/or maneuverability for traversing tortuous paths, such as paths within the neurovascular system.

In one aspect, the present disclosure relates to methods and systems for selectively adjusting the flexibility of a segment of a lumen (e.g., delivery element, tubular extension of a removable hub system, catheter, etc.). A delivery element such as a catheter, in some implementations, includes a grind or cut pattern through at least a portion of the thickness of the material to enable increased flexibility. The grinding or cutting pattern, for example, may be applied as a graduated spiral, where increased density of the cutting pattern corresponds to a relative increase in flexibility. In the example of a metal hypotube, the metal lumen may be spiral cut and coated with a flexible material, such as a polymer coating. The spiral cut, for example, may transform the rigid metal hypotube to a flexible coil, where the flexibility depends on the density of the spiral cut (e.g., a number of spirals per unit length). In another example, applying an increasingly dense spiral cut may create a transition segment, transitioning from a rigid hypotube section to a flexible section. The flexible section, for example, may be configured to interface with a flexible delivery element, such as a catheter. In the example of a polymer catheter, a segment of the catheter may be ground down in a constant or patterned thinning to increase flexibility. The grinding may be applied in a directional fashion, such that the resultant material is “trained” to flex in a particular direction (e.g., a steerable catheter end). In illustration, the grinding may be performed such that a selected side (e.g., approximately one sixth to one half of the circumference) of the catheter is thinned in a consistent or graduated manner along a predetermined length to encourage increased flexibility in the direction opposite of the selected side.

In some embodiments, a method for using a removable hub system includes disconnecting an instrumentation hub portion of the removable hub system from a locking shaft element of the removable hub system, and removing the locking shaft element from around the proximal end of an instrumentation delivery element (e.g., catheter) while a flexible extending element (e.g., guidewire) is fed through the instrumentation delivery element with a distal end disposed at a procedural site in a patient. The method may include removing the instrumentation delivery element while maintaining the flexible extending element in position. The method may include, prior to removing removable hub system, fixing a position of the flexible extending element with an external securing system such that the distal end of the flexible extending element is prevented at least from moving backwards away from the procedural site. The method may include replacing the instrumentation delivery element with a different instrumentation delivery element having a proximal end shaped for enclosure by the locking shaft element of the removable hub system. The method may include securing the locking shaft element around the circumference of the proximal portion of the different instrumentation delivery element and connecting the instrumentation hub portion of the removable hub system to the locking shaft element of the removable hub system. If the position of the flexible extending element was previously fixed, the method may include freeing the flexible extending element for movement.

The foregoing general description of the illustrative implementations and the following detailed description thereof are merely exemplary aspects of the teachings of this disclosure, and are not restrictive.

### BRIEF DESCRIPTION OF THE DRAWINGS

The accompanying drawings, which are incorporated in and constitute a part of the specification, illustrate one or more embodiments and, together with the description, explain these embodiments. The accompanying drawings have not necessarily been drawn to scale. Any values dimensions illustrated in the accompanying graphs and figures are for illustration purposes only and may or may not represent actual or preferred values or dimensions. Where applicable, some or all features may not be illustrated to assist in the description of underlying features. In the drawings:

FIG. 1 is a cross-sectional view illustrating a portion of a first example catheter having a central lumen connected at the proximal end to a flexible extending element of a first example removable hub;

FIGS. 2A and 2B illustrate the proximal end of the catheter of FIG. 1 with and without the first example removable hub system;

FIG. 3 illustrates an example locking shaft element for securing a removable hub system to the proximal end of a flexible shaft;

FIG. 4A through FIG. 4C illustrate an example versatile catheter system with a second example removable hub system;

FIG. 5 illustrates the proximal end of a second example flexible shaft including an attachment element for releasably receiving a removable hub system;

FIG. 6A and FIG. 6B illustrate additional attachment element designs for connecting a removable hub system to a flexible shaft;

FIG. 7 illustrates an example detachable fluid management system for coupling to the proximal end of a flexible shaft;

FIG. 8 is a third example flexible shaft including a curved portion for selectably locking a position of a flexible extending element within an internal lumen of the flexible shaft;

FIG. 9A and FIG. 9B illustrate a first example wedge element for locking a position of a flexible extending element relative to a flexible shaft;

FIG. 9C illustrates a second example wedge element;

FIG. 10A and FIG. 10B illustrate an example sleeve-type guidewire position locking mechanism for use with a flexible shaft;

FIG. 11A and FIG. 11B illustrate a third example removable hub system;

FIG. 12A through 12D illustrate example retention features for releasably coupling a removable hub apparatus with a flexible shaft.

FIG. 13A through FIG. 13C illustrate a portion of an example removable hub system including a flared tubular member for mating with a flexible delivery element;

FIG. 14 illustrates an example instrumentation system including a removal hub system, a flexible delivery element, and an extension delivery element; and

FIG. 15A through FIG. 15C illustrate example material treatments for selectively adjusting flexibility of segments of a flexible delivery element or other tubular member.

### DETAILED DESCRIPTION OF ILLUSTRATIVE EMBODIMENTS

The description set forth below in connection with the appended drawings is intended to be a description of various, illustrative embodiments of the disclosed subject matter. Specific features and functionalities are described in connection with each illustrative embodiment; however, it will be apparent to those skilled in the art that the disclosed embodiments may be practiced without each of those specific features and functionalities.

Reference throughout the specification to “one embodiment” or “an embodiment” means that a particular feature, structure, or characteristic described in connection with an embodiment is included in at least one embodiment of the subject matter disclosed. Thus, the appearance of the phrases “in one embodiment” or “in an embodiment” in various places throughout the specification is not necessarily referring to the same embodiment. Further, the particular features, structures or characteristics may be combined in any suitable manner in one or more embodiments. Further, it is intended that embodiments of the disclosed subject matter cover modifications and variations thereof.

It must be noted that, as used in the specification and the appended claims, the singular forms “a,” “an,” and “the” include plural referents unless the context expressly dictates otherwise. That is, unless expressly specified otherwise, as used herein the words “a,” “an,” “the,” and the like carry the meaning of “one or more.” Additionally, it is to be understood that terms such as “left,” “right,” “top,” “bottom,” “front,” “rear,” “side,” “height,” “length,” “width,” “upper,” “lower,” “interior,” “exterior,” “inner,” “outer,” and the like that may be used herein merely describe points of reference and do not necessarily limit embodiments of the present disclosure to any particular orientation or configuration. Furthermore, terms such as “first,” “second,” “third,” etc., merely identify one of a number of portions, components, steps, operations, functions, and/or points of reference as disclosed herein, and likewise do not necessarily limit embodiments of the present disclosure to any particular configuration or orientation.

Furthermore, the terms “approximately,” “about,” “proximate,” “minor variation,” and similar terms generally refer to ranges that include the identified value within a margin of 20%, 10% or preferably 5% in certain embodiments, and any values therebetween.

All of the functionalities described in connection with one embodiment are intended to be applicable to the additional embodiments described below except where expressly stated or where the feature or function is incompatible with the additional embodiments. For example, where a given feature or function is expressly described in connection with one embodiment but not expressly mentioned in connection with an alternative embodiment, it should be understood that the inventors intend that that feature or function may be deployed, utilized or implemented in connection with the alternative embodiment unless the feature or function is incompatible with the alternative embodiment.

The present disclosure discusses a variety of solutions, many of which are interoperable with a basic catheter, microcatheter, and/or guidewire (generally referred to as a “flexible shaft” or “delivery element” herein), configured to extend the utility and interoperability of the catheter design for enabling procedure stacking and/or switching while maintaining the position of a flexible extending element (e.g., microcatheter or guidewire) at a procedural site. The flexible shaft, for example, may be a commonly used catheter available on the market from a variety of suppliers.

In some embodiments, the utility and interoperability of a basic or customized flexible shaft design is extended through including removable connectors, such as fluid hub systems, instrument hub systems, and/or other proximally-coupled control elements used during a medical procedure for introducing medical instruments and/or fluids at a procedure site. The removable connectors, for example, may be inter-operable with the flexible shaft and/or a shaft mating element such that the removable connectors may be exchanged during a procedure to allow for different therapeutic steps to be performed by a medical professional.

The removable connectors and/or other elements of a proximally-coupled control element system, in some implementations, can be treated with materials to assist in ease of connection and/or removal. For example, various elements that may be included in a proximally-coupled control element system can be coated to improve lubricity with hydrophilic and/or hydrophobic materials. In another example, the various elements of the proximally-coupled control element system may be formed of or include an outer layer of a non-stick material such as a polytetrafluoroethylene (e.g., Teflon™ by Chemours Company of Wilmington, Delaware).

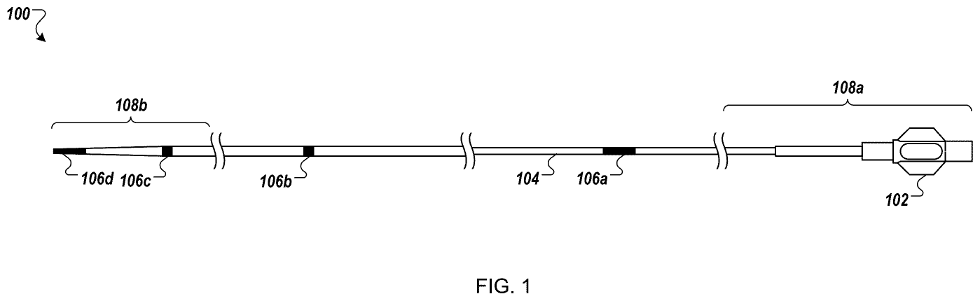

In some embodiments, the flexible shaft of various embodiments described herein is an improved catheter design configured for use in complex vascular procedures and offering enhanced control, visibility, and overall effectiveness. The catheter, for example, may be developed for insertion into both arterial and venous systems, thereby offering increased versatility. Turning to FIG. 1, a cross-sectional view of an example versatile catheter 100 illustrates a hub 102 at the proximal end 108a and a flexible shaft 104 connected to and extending from the hub 102. The flexible shaft 104 (e.g., catheter or microcatheter), for example, may include at least one lumen extending therethrough.

In some implementations, the flexible shaft 104 is designed for coaxial delivery, such that other medical devices and/or therapies may be provided both through the at least one lumen of the versatile catheter 100 as well as over the flexible shaft 104 of the versatile catheter 100. The dimensions of the flexible shaft 104, in some examples, can be about 100 cm to 200 cm in length, or preferably about 120-170 cm in length, with a diameter within a range from 2 to 12 French (e.g., 0.6 mm to 4.0 mm). In a particular example, the flexible shaft may be 140 cm long and 7 Fr in diameter. As described in greater detail below, regions of the flexible shaft 104 may vary in interior and/or exterior diameter.

The hub 102, in some implementations, is a removable feature that is releasably attached to the proximal end of the flexible shaft 104. The hub 102 may be released, for example, to introduce additional medical devices over the versatile catheter 100. In another example, the hub 102 may be exchanged for another hub design, depending on the needs of a current procedure. The hub 102, in some examples, may include a fluid management feature (e.g., rotating hemostasis valve, port, or other mechanism) for use in introducing and/or removing fluid. In some examples, the hub 102 may be used to introduce an imaging contrast, introduce a therapeutic agent, provide aspiration of internal fluid, and/or prevent blood loss. For example, the hub 102, when added to the proximal end of the flexible shaft 104, may be configured to seal the passage provided by the lumen of the flexible shaft 104.

In some embodiments, the proximal end of the flexible shaft 104 is composed of one or more rigid or semi-rigid materials to provide for connection between the flexible shaft 104 and the hub 102. For example, a proximal section of the flexible shaft 104 designed to enable interfacing with the hub 102 may be composed of metals and/or hard polymers. Alloys, such as stainless steel and/or Nitinol®, may be used, in some implementations, to further enhance the shape memory of a polymer. In further embodiments, the proximal section may be constructed using a braid. A braid, for example, can be made from an alloy, such as stainless steel or Nitinol®. The braid can be bare or coated with a soft elastomeric polymer. The material selection of the proximal section of the flexible shaft 104 may depend, in part, on a type of connection made between the flexible shaft 104 and the hub 102 (e.g., frictional, threaded, etc.). Illustrative examples are described below.

In some embodiments, the flexible shaft 104 is sufficiently flexible to bend along the patient's vasculature or other series of vessels while having a rigidity that resists kinking or folding. For example, a durometer D of the material(s) composing the flexible shaft 104 may be greater than 50. In another example, the material(s) composing the flexible shaft 104 may exhibit a deflection force (e.g., the force required to deflect the section of material to 90° as measured by a force gauge) within a range of 6.2 N to 12.2 N. The deflection force values may be measured in accordance with the Coronary, Peripheral, and Neurovascular Guidewires—Performance Tests and Recommended Labeling guidance document published by the Food and Drug Administration in October 2019. For example, the deflection force value may be measured when the device is held at 20 mm from the distal end.

Portions of the versatile catheter 100, in some embodiments, are formed from one or more biocompatible materials, including, for example, metals, such as stainless steel or alloys, e.g., Nitinol®, or polymers such as polyether-amide block co-polymer (PEBAX®), nylon (polyamides), polyolefins, polytetrafluoroethylene, polyesters, polyurethanes, polycarbonates, mixtures thereof, copolymers thereof or other suitable biocompatible polymers.

In some embodiments, the flexible shaft 104 is composed of imaging-compatible (e.g., MRI-compatible) materials, and radiopaque markings 106a-d are provided for identifying positioning of the versatile catheter 100. Radiopacity, in some examples, can be added to the versatile catheter 100 using platinum-iridium and/or platinum-tungsten. The radiopaque markings 106a-d, in some implementations, are added to an exterior of the material of the flexible shaft 104 (e.g., a radiopaque sleeve). In other implementations, the radiopacity is achieved using through radio-pacifiers, such as barium sulfate, bismuth trioxide, bismuth subcarbonate, powdered tungsten, powdered tantalum or the like, added to the polymer of the flexible shaft 104.

The radiopaque markings 106a-d, in some embodiments, are disposed in part at transition locations along the flexible shaft 104. For example, as illustrated radiopaque marker 106c is positioned prior to a reduction in diameter that tapers to a tip of the flexible shaft 104. The tip, similarly, is identified using the radiopaque marker 106d. The radiopaque markers 106c, 106d positioned prior to and upon the tip of the flexible shaft 104, for example, may be used to confirm anatomic placement of the flexible shaft and/or instrumentation delivered therefrom.

Generally, different sections of the versatile catheter 100 can be formed from different materials from other sections. Further, certain sections of the versatile catheter 100 can be composed from multiple materials at different locations and/or at a particular location. In some embodiments, selected sections of the versatile catheter 100 are formed with materials to introduce desired stiffness/flexibility for the particular section of the versatile catheter 100. For example, fittings and the like can be formed from a more rigid or semi-rigid material, such as one or more metals and/or one or more polymers. In another example, it may be desirable to form the distal section 108b of the versatile catheter 100 or a portion thereof (e.g., at or near the distal end of the versatile catheter 100) from an elastomeric polymer, such as suitable polyurethanes, polydimethylsiloxane and/or polytetrafluoroethylene, for increased flexibility. In illustration, a section of the versatile catheter 100 having increased flexibility may be composed of a material with a Shore A Durometer D value of no more than 45. Enhanced flexibility, for example, may improve tracking properties for placement of the versatile catheter 100.

In some embodiments, the flexible shaft 104 is mainly or fully composed of a thermoplastic polymer with embedded metal wire. Suitable polymers include, for example, polyamides (nylons). The wire can be braided, coiled, or otherwise placed over a polymer tubing liner with some tension. A polymer jacket may then be placed over the top. Upon heating over the softening temperature of the polymer and subsequent cooling, the wire becomes embedded within the polymer. The polymer liner and polymer jacket can be of the same or different materials. Suitable wire includes, for example, flat stainless-steel wire and/or tungsten wire. The wire, for example, may be configured to add additional mechanical strength while maintaining appropriate amounts of flexibility.

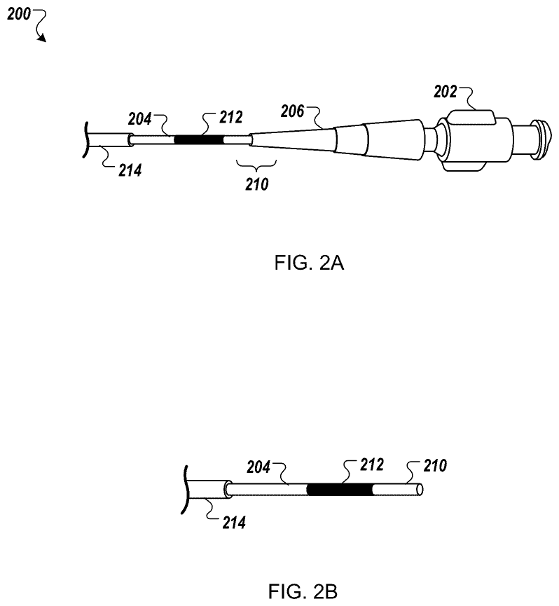

FIG. 2A illustrates an example removable hub system 200 for a catheter such as the versatile catheter system 100 of FIG. 1. For example, as illustrated, the removable hub system 200 is releasably attached to the proximal end of a flexible shaft 204 (e.g., the flexible shaft 104 of FIG. 1). As shown in FIG. 2B, a proximal end 210 of the flexible shaft 204 includes no special fitting. The removable hub system 200, for example, may be frictionally attached to the proximal end 210 of the flexible shaft 204.

Turning to FIG. 2A, in some embodiments, the removable hub system 200 includes a shaft mating element 206 that couples, at one end, to the proximal end 210 of the flexible shaft 204 and, at the other end, with a hub element 202 of the removable hub system 200. Although illustrated as having a shortened length, in other embodiments, the proximal end 210 of the flexible shaft 204 may extend further within the shaft mating element 206. The shaft mating element 206 may include a female connection mechanism, such as a flexible seal, thread grooves, or channels, that mates with a male connection mechanism of the hub element 202. Conversely, the male and female connection mechanisms may be swapped, in certain embodiments. The mated connection mechanisms, in some examples, can include a threaded connection, one or more channels (straight, twist-locking channels, etc.), a frictional seal, a pop or snap fitting, or a deformable compression fitting. On the opposite end, the shaft mating element 206, in one example, may be frictionally retained around a proximal end 210 of the flexible shaft 204 (e.g., using a sticky surface finish, a rough surface finish, etc.). In another example, the shaft mating element 206 may include a deformable material for compressively fitting around the proximal end 210 of the flexible shaft 204 (e.g., rubber, silicone, polyvinyl chloride (PVC) or polyurethane (PU) foam, etc.). In a further example, the shaft mating element 206 may clamp to the proximal end 210 of the flexible shaft 204, for example via a hinged or flexible closure.

As illustrated in FIG. 2A and FIG. 2B, the flexible shaft 204 includes a marker 212 (e.g., a radiopaque marker or other marker). The marker 212, for example, may be used, in some embodiments, to align the flexible shaft 204 with the shaft mating element 206. In illustration, the marker 212, in an embodiment not illustrated, may be visually aligned at a distal end of the shaft mating element 206 to ensure consistent and dependable retention of the proximal end 210 of the flexible shaft 204 within the shaft mating element 206.

The portion of the flexible shaft 204 illustrated in FIG. 2A and FIG. 2B includes a segment 214 with increased diameter in comparison to a proximate segment having the marker 212. The segment 214, for example, may be disposed in a location configured for gripping of the flexible shaft 204 to improve manipulation by a medical professional when mating the flexible shaft 204 with the shaft mating element 206. In other embodiments, the flexible shaft 204 may have a consistent diameter or a tapered (e.g., graduated) diameter. In some implementations including varying diameters, an internal diameter for the flexible shaft 204 remains consistent.

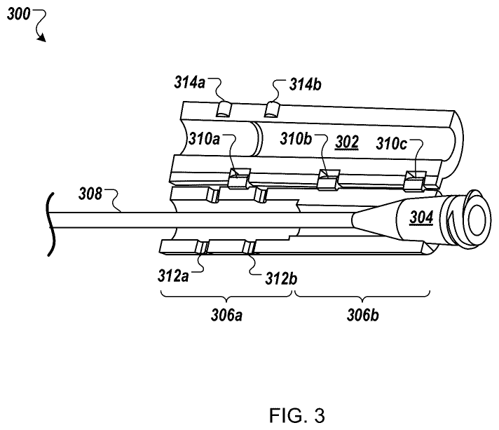

Turning to FIG. 3, a portion of a removable hub system 300 is illustrated, including an example locking element 302 and a shaft mating element 304. As illustrated, the locking element 302 is configured to close around a portion of the proximal end of a flexible shaft 308 and a portion of the shaft mating element 304 that is coupled with (e.g., frictionally inserted into) the flexible shaft 308 to retain the mated connection between the shaft mating element 304 and the flexible shaft 308. The locking element 302, as illustrated, includes two pieces connected by a set of hinges 310. A detent 312 and protrusion 314 snap fit, as shown, may be used to releasably lock the locking element 302 into position. In other embodiments, another locking mechanism may be used, such as, in some examples, a slideable lock within a mated groove or a pin and channel lock.

In some embodiments, an inner cavity of the locking element 302 is graduated or otherwise formed around the dimensions of the shaft mating element 304 and/or the flexible shaft 308. For example, a distal portion 306a of the interior surface of the locking element 302 may have a wider opening to accommodate flexing of the flexible shaft 308, while a proximal portion 306b of the interior surface of the locking element 302 may be dimensioned to closely form to the shape of the portion of the shaft mating element 304 captured by the locking element 302.

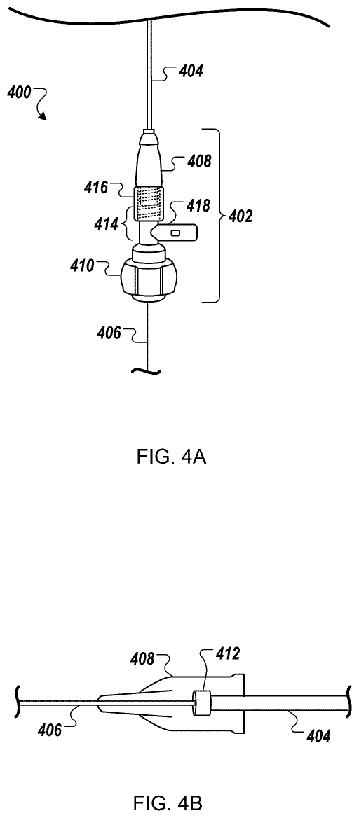

Turning to FIG. 4A through FIG. 4C, in some implementations, a versatile catheter system 400 includes a removable hub system 402 releasably connected to a flexible shaft 404 including at least one lumen for passing an extending element 406 (e.g., guidewire). The removable hub system 402, for example, may include a shaft mating element 408 coupled to a hub element 410. The hub element 410, for example, may include a fluid control port 418 mounted to a tubular extension 414 which, at its distal end, includes one or more mating features 416 (e.g., a screw or luer lock) to mate with the shaft mating element 408.

Turning to FIG. 4B, in some implementations, the flexible shaft 404 includes an expansion element 412 creating a ledge or step outward in circumference of the flexible shaft 404. The expansion element 412, in some examples, may be integral with or fused to the flexible shaft 404. In some examples, the expansion element 412 may be three-dimensionally printed onto the flexible shaft 404, adhesively added to the flexible shaft 404, and/or produced integrally as part of the material of the flexible shaft 404 during manufacture. In other embodiments, the expansion element 412 is removably coupled to the flexible shaft 404. The expansion element 412, for example, may be slid snugly onto the flexible shaft 404. In some examples, the expansion element 412 may be molded, cast, and/or three-dimensionally printed. The expansion element 412, in some examples, may be formed of a rigid or semi-rigid material such as a metal, metal alloy, and/or polymer. In some implementations, the expansion element 412 includes compressible or flexible material, for example to assist in being frictionally retained. As illustrated, the expansion element 412 may be designed to be captured in a cavity of the shaft mating element 408 such that the flexible shaft 404 is prevented from moving in its lengthwise direction. The expansion element 412, as illustrated, is generally rounded. In other embodiments, the expansion element 412 may have edges (e.g., square, hexagonal, etc.). The edges, for example, may limit the ability of the flexible shaft 404 to rotate within the shaft mating element 408.

Turning to FIG. 4C, in some implementations, the shaft mating element 408 of FIG. 4B is designed as two pieces 408a, 408b, such that the two pieces may lock around the proximal end of the flexible shaft 404, including around the expansion element 412. The two pieces, for example, may be hinged as illustrated in the shaft locking element 302 of FIG. 3. In another example, the two pieces may releasably connect on both sides, for example using two sets of a protrusion and detent mating elements.

In some implementations, the hub 410 couples to the shaft mating element 408 using a standard male luer lock fitting 416, as illustrated. The luer lock fitting 416, for example, may allow for leak-free introduction of fluids to the flexible shaft 404 via the fluid port 418 of the hub 410.

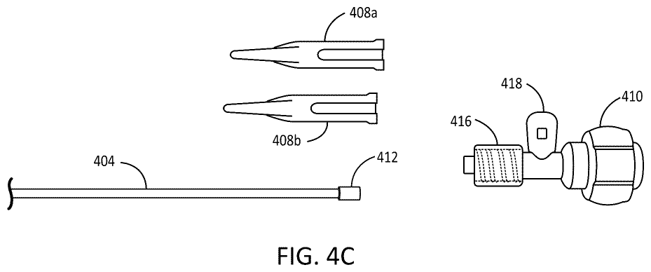

FIG. 5 illustrates the proximal end of an example flexible shaft 500 including an attachment element 502 for releasably receiving a removable hub system, such as the removable hub 102 of FIG. 1 and/or the removable hub system 200 of FIG. 2A and FIG. 2B. The attachment element 502, for example, may be designed as an expansion element, for example to be captured by a shaft mating element such as the shaft mating element 408 of FIG. 4A through FIG. 4C. The attachment element 502, in some embodiments, is permanently attached to the flexible shaft 500. In some examples, the attachment element 502 may be three-dimensionally printed onto the flexible shaft 500, adhesively added to the flexible shaft 500, and/or produced integrally as part of the material of the flexible shaft 500 during manufacture. In other embodiments, the attachment element 502 is removably coupled to the flexible shaft 500. The attachment element 502, for example, may be slid snugly onto the flexible shaft 500. In some examples, the attachment element 502 may be molded, cast, and/or three-dimensionally printed. The attachment element 502, in some examples, may be formed of a rigid or semi-rigid material such as a metal, metal alloy, and/or polymer. In some implementations, the attachment element 502 includes compressible or flexible material, for example to assist in being frictionally retained.

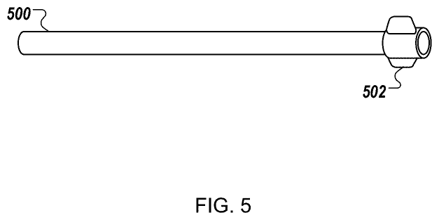

FIG. 6A and FIG. 6B illustrate additional attachment element designs for connecting a removable hub system to a flexible shaft. Turning to FIG. 6A, a shaft mating element 600 includes a distal end connection mechanism 608 for releasably connecting the shaft mating element 600 to a corresponding proximal end connection mechanism 606 of a flexible shaft 604. To enable a medical professional to grasp and manipulate the shaft mating element 600 during a medical procedure, in some embodiments, the shaft mating element 600 includes extensions or wings 602a,b. For example, as illustrated, the male threads connection mechanism 606 of the proximal end of the flexible shaft 604 are configured to twistably connect with the female thread connection mechanism 608 of the shaft mating element 600. The extensions 602a, b, further to the example, may provide leverage to a medical practitioner for twisting the shaft mating element 600 onto and off of the end connection mechanism 606 of the flexible shaft 604. On the opposite end of the shaft mating element 600, a luer connection mechanism 610 may include standard rotating threads for connecting with a luer lock mechanism of a hub element, such as the luer lock connector 416 of the hub element 410 of FIG. 4C.

Turning to FIG. 6B, a shaft mating element 620 for releasably connecting a removable hub system to a flexible delivery element includes a distal end connection mechanism 622 for capturing an expansion element 626 (e.g., such as the expansion element 412 of the flexible shaft 404 of FIG. 4B and FIG. 4C and/or the connection element 502 of FIG. 5) of a flexible shaft 624. The distal end connection mechanism 622, in some examples, can include one or more flexing members 632 configured to flex outward and over the expansion element 626. The flexing member(s) 632 of the distal end connection mechanism 622, for example, may exert a retention force against and/or distal to the expansion element 626 of the flexible shaft 624. To enable a medical professional to grasp and manipulate the shaft mating element 620 during a medical procedure, in some embodiments, the shaft mating element 620 includes a gripping region 628 including a textured surface and/or extending features configured to improve handling of the shaft mating element 620. In some examples, the gripping region 628 may include a rough and/or tacky (e.g., rubberized) surface finish, a series of raised ribs, and/or an arrangement of protrusions. The raised surface features, such as ribs and/or protrusions, may be flexible and/or treated with a surface finish (e.g., rough or tacky, etc.) to further enhance handling. On the opposite end of the shaft mating element 620, in some embodiments, a female luer connection mechanism 630 (e.g., a two-part compression fit mechanism) is provided for connecting with standard rotating threads of a luer lock mechanism of a hub element, such as the luer lock element 416 of the hub element 410 of FIG. 4C.

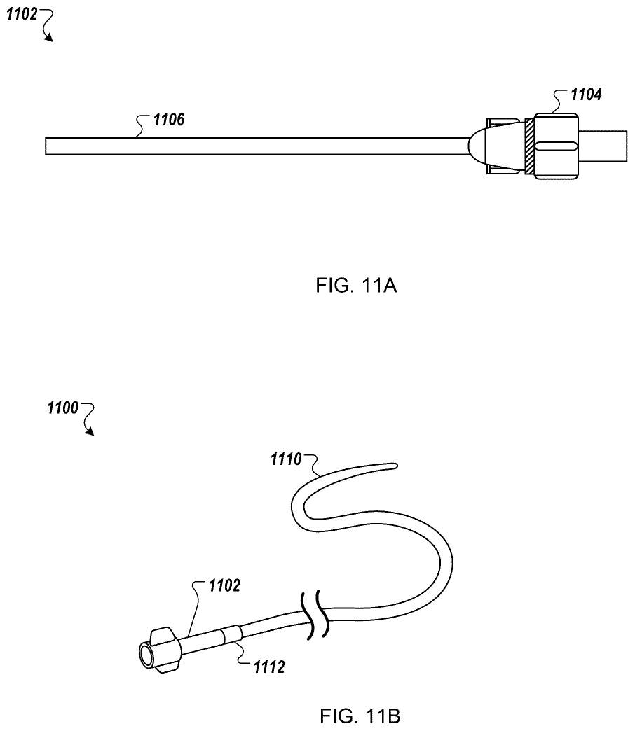

Turning to FIG. 11A and FIG. 11B, in some implementations, an example removable hub system 1100 for releasably coupling with a flexible shaft includes a removable hub apparatus 1102 including a hub member 1104 (e.g., fluid management hub and/or instrument management hub, such as the hub 102 of FIG. 1) and a tubular member 1106 extending in a distal direction from the hub member 1102. The removable hub apparatus 1102 may be released from the proximal end of a flexible shaft 1110, for example, to introduce additional medical devices over the flexible shaft 1110 (e.g., the versatile catheter 100 of FIG. 1). The hub member 1104, in some examples, may include a fluid management feature (e.g., rotating hemostasis valve, port, or other mechanism) for use in introducing and/or removing fluid. In some examples, the hub member 1104 may be used to introduce an imaging contrast, introduce a therapeutic agent, provide aspiration of internal fluid, and/or prevent blood loss. For example, the removable hub apparatus 1102, when added to the proximal end of the flexible shaft 1110, may be configured to seal the passage provided by the lumen of the flexible shaft 1110.

Turning to FIG. 11A, a cross-sectional view illustrates the removable hub apparatus 1102. In some implementations, the hub member 1104 is integrated with, fused to, or otherwise permanently attached to the tubular member 1106. In other implementations, the hub member 1104 is releasably connected to the tubular member 1106. For example, a luer lock mechanism may be used to connect the hub member 1104 to the tubular member 1106. The tubular member 1106 may be formed of rigid or semi-rigid material, such as a hypotube. The tubular member 1106 may be include multiple deflection force values, for example increasing in flexibility toward a distal end of the tubular member 1116 to better mate with the flexible shaft 1110, as illustrated in FIG. 11B.

Turning to FIG. 11B, in some implementations, the removable hub apparatus 1102 releasably connects to the flexible shaft 1110 at a mating segment 1112 of the flexible shaft 1110. The mating segment 1112, for example, may include one or more retention features to enable coupling of the flexible shaft 1110 with the removable hub apparatus 1102.

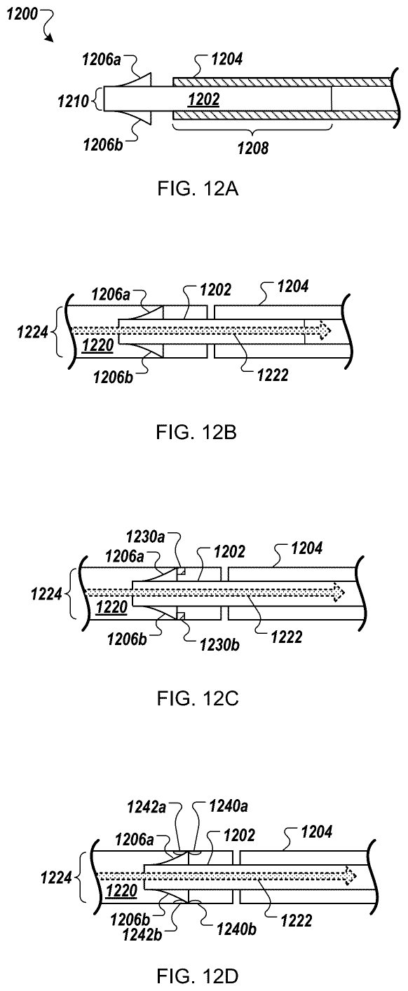

FIG. 12A through 12D illustrate example retention features for releasably coupling a removable hub apparatus with a flexible shaft. The retention features, for example, may include one or more conical members, wedge-shaped members, fin-shaped members, and/or barb-shaped members disposed at an end of one of the removable hub apparatus or the flexible shaft and configured to frictionally engage with an interior lumen and/or a coordinating ridge or ramp feature within the lumen of the mating member (e.g., removable hub apparatus or flexible delivery element). Although described in relation to the removable hub apparatus and the flexible shaft, in other embodiments, the retention features may be provided for releasably mating the flexible shaft with a flexible shaft extension. For example, the retention features may be pushed into a lumen of a mating flexible shaft when connecting, and the flexible shaft coupled to the retention features may be pulled apart from the mating flexible shaft (e.g., removable hub apparatus) to disconnect the two pieces from each other. In other embodiments, a twisting motion may be required to lockably mate the two pieces to each other and, conversely, disconnect the mating between the two pieces of equipment.

Turning to FIG. 12A, an end section of a first apparatus 1200 (e.g., catheter, removable hub apparatus, etc.) may include a mating segment 1202 composed of a rigid or semi-rigid material and extending beyond a connecting segment 1204 partially overlapping 1208 with the mating segment 1202 and affixed to the mating segment. The connecting segment 1204, for example, may be formed of a polymer material, and the mating segment 1202 may be formed of one or more metals, such as a hypotube. The connecting segment 1204, in some examples, may be adhered, welded, or otherwise secured to the mating segment 1202. The connecting segment 1204, in illustration, may be a conventional catheter or microcatheter or another delivery element as described herein.

In some implementations, the mating segment 1202 includes one or more retention features 1206 (e.g., barbs) arranged on an exterior surface of a lumen 1210 of the mating segment 1202. The retention features 1206, for example, may be integrated with (e.g., printed of a same material, manufactured or formed of a same material, etc.) the lumen 1210 of the mating segment 1202. In another example, the retention features 1206a may be attached to the exterior surface of the lumen 1210. In illustration, the retention features 1206 may be formed of stainless steel and welded onto the surface of a hypotube. In another example, the retention features 1206 may be formed from one or more polymer materials and printed onto, welded to, or adhered to the surface of the lumen 1210.

Turning to FIG. 12B, in some implementations, the mating segment 1202 is inserted into an end of a second apparatus 1220 (e.g., catheter removable hub apparatus, etc.), thereby placing the first apparatus 1200 in fluid communication 1222 with the second apparatus 1220. As illustrated, the retention features 1206 abut an interior surface of a lumen 1224 of the second apparatus 1220. The retention features 1206, for example, may frictionally retain position against the interior surface of the lumen 1224.

In some implementations, at least an interior surface of the lumen 1224 is formed of a flexible or semi-flexible material, such that retention features 1206 formed of a rigid material push into the flexible or semi-flexible material, thereby frictionally retaining position. In other implementations, the retention features 1206 are formed of a flexible or semi-flexible (e.g., compressible) material such that the retention features 1206 deform when inserted into the lumen 1224 of the second apparatus 1220, thereby frictionally retaining position against the interior surface of the lumen 1224. Conversely, pulling the first apparatus 1200 away from the second apparatus 1220 may overcome the frictional force and result in separation of the first apparatus 1200 from the second apparatus 1220.

As illustrated in FIG. 12C and FIG. 12D, in some implementations, the interior surface of the lumen 1224 includes one or more corresponding retention features (1230, 1240, 1242) designed to interface with the one or more retention features 1206. The corresponding retention feature(s), for example, may be designed to capture or limit the movement of the retention features 1206. The corresponding retention feature(s) 1230, for example, may be formed of a semi-flexible or flexible material, such as, in some examples, rubber, silicone, PVC, or PU foam. In the circumstance of a semi-flexible or flexible retention feature(s) 1206, the corresponding retention feature(s) may be formed of rigid or semi-rigid material, such as stainless steel or one or more polymers. The corresponding retention feature(s) 1230 may be molded or formed as part of the lumen 1224.

Turning to FIG. 12C, first example corresponding retention feature(s) 1230 include one or more protrusions, such as a ridge. The barbed retention features 1230, for example, may push over the corresponding retention feature(s) 1230 which act as a bumper, limiting distal motion of the first apparatus 1200, thereby supporting connection between the first apparatus 1200 and the second apparatus 1220. Although illustrated as substantially angular, in other embodiments, the retention feature(s) 1230 may be curved or partially curved (e.g., curved at a distal end to assist in insertion of the mating segment 1202 and straight at a proximal end to provide greater support in retaining position of the retention feature(s) 1206).

For example, turning to FIG. 12D, the retention feature(s) 1206, upon insertion into the lumen 1224, may press over first corresponding retention feature(s) 1240 (e.g., a partial or full diameter ridge or ledge) and stop upon engaging second corresponding retention feature(s) 1242. Although described as first and second corresponding retention feature(s) 1240, 1242, in other embodiments, a dual-protrusion corresponding retention feature may be provided within the lumen 1224. In some embodiments, the second corresponding retention feature(s) 1242 may be formed of a more rigid material or otherwise be designed to prevent insertion beyond the second corresponding retention feature(s) 1242.

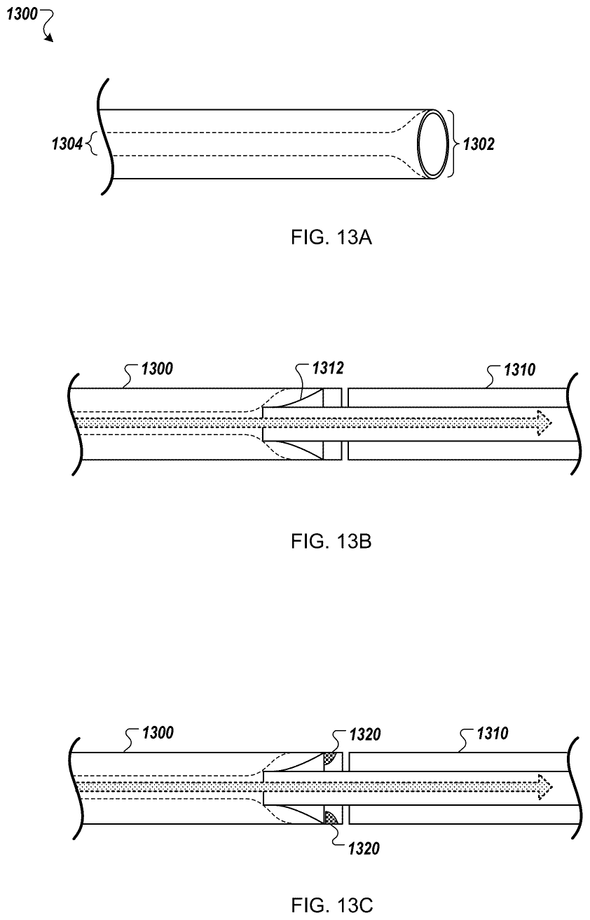

FIG. 13A and FIG. 13B illustrate a portion of an example removable hub system including a flared tubular member for mating with a flexible delivery element. Although described as part of the removable hub system, similar connecting features may be used to interface between a flexible delivery element and an extension element, such as a second catheter length to enable a longer trajectory of treatment and/or to couple/decouple different flexibility and/or styles of catheter depending upon the purpose and/or route of the procedure.

Turning to FIG. 13A, a tubular member 1300 of a removable hub apparatus may include a widened distal opening 1302, providing the benefit of a larger external diameter for easier manipulation. Further, the wider distal opening 1302 may be used to guide the proximal end of a mating delivery element for ease of connection. For example, as illustrated in FIG. 13B, a mating component 1312 of a delivery element 1310 may be more easily guided into the internal lumen of the tubular member 1300 vial the widened distal opening 1302. The mating component 1312, for example, may include one or more mating features, such as the mating features described in relation to the retention feature(s) 1206 of the apparatus 1200 of FIG. 12A through FIG. 12D.

The narrower fluid passage 1304 proximal to the widened distal opening 1302, in some implementations, allows for creating a fluid seal between an end of the mating component 1312 and the interior lumen of the tubular member 1300. For example, as illustrated in FIG. 13B, the proximal end of the mating component 1312 abuts against a diameter at a narrower end of a funnel shape leading to the wider distal opening 1302. Although illustrated as a funnel shape, in other embodiments, the transition between the wider distal opening 1302 and the narrower fluid passage 1304 may have a different shape, such as an angular transition, a concave curved transition (as illustrated), or a convex curved transition of various angles.

To encourage a fluid seal between the interior lumen of the tubular member 1300 and the mating component 1312 of the delivery element 1310, in some implementations, the surface of a proximal edge of the mating component 1312 and/or the interior surface of at least a portion of the lumen of the tubular member 1300 may be treated with a sealing material such as, in some examples, rubber, silicone, PVC, or PU foam. In certain embodiments, one or more corresponding mating members may affect a seal, such as a ridge member described in relation to FIG. 12C. Turning to FIG. 13C, for example, the inner lumen of the tubular member 1300 may include a sealing retainer 1320 to create a fluid seal between the tubular member 1300 and the delivery element 1310.

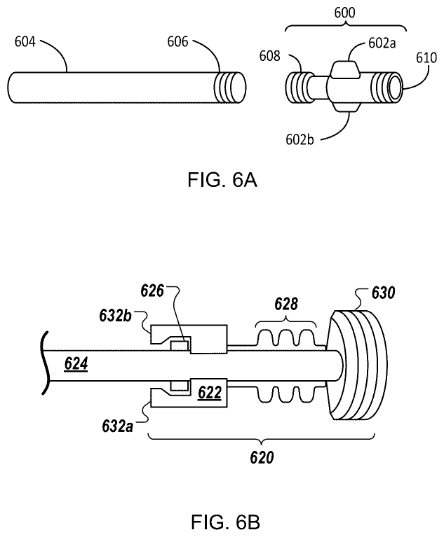

FIG. 7 illustrates an example detachable fluid management system 700 for coupling to the proximal end of a flexible shaft 702. The detachable fluid management system 700, for example, may be configured to interoperate with a shaft mating element and/or shaft locking element (e.g., mating element 206 of FIG. 2A, locking element 302 of FIG. 3, mating element 408 of FIG. 4A to FIG. 4C, coupling element 502 of FIG. 5, mating element 600 of FIG. 6A, mating element 628 of FIG. 6B, etc.) to couple the detachable fluid management system 700 to the flexible shaft 702. A practitioner, for example, may be able to exchange a hub element (e.g., the hub 202 of FIG. 2A or hub 410 of FIG. 4A) for the detachable fluid management system 700 using a locking and/or mating element while performing a therapeutic procedure on a patient.

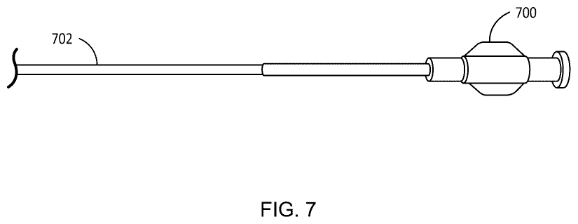

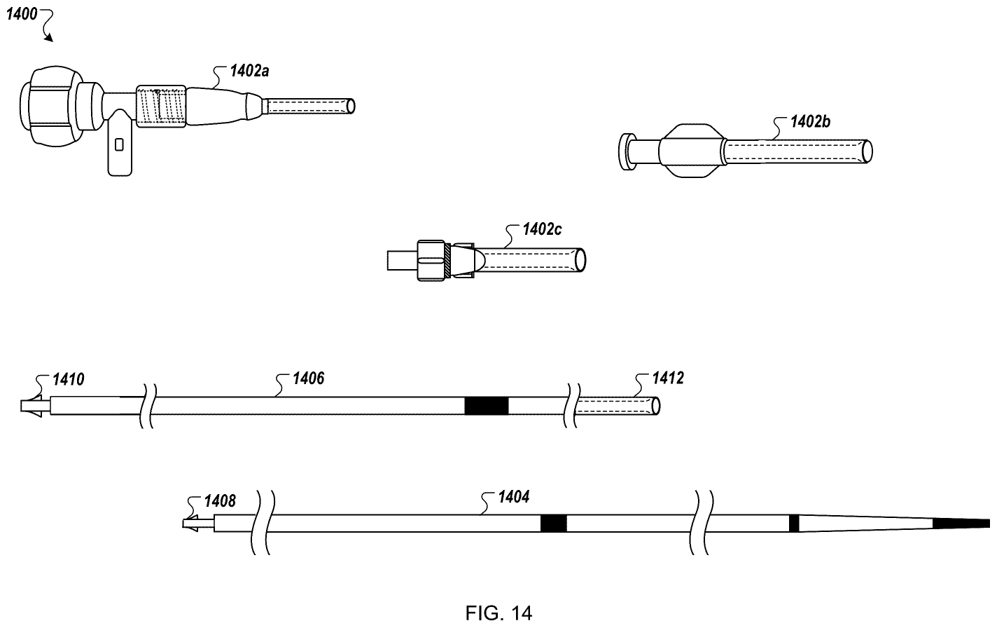

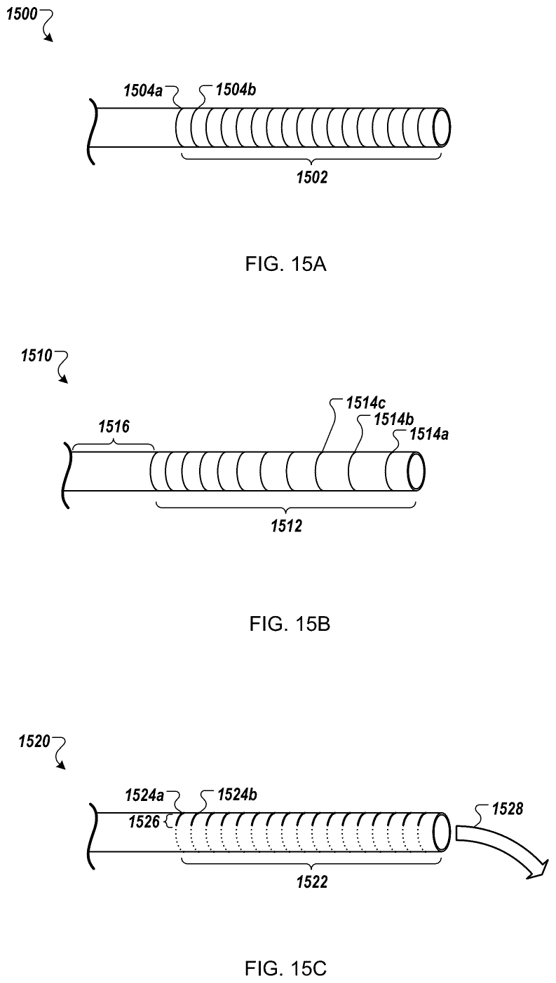

FIG. 14 illustrates an example instrumentation system 1400 with a removal hub system 1402 including a set of three swappable instrument hubs 1402a, 1402b, and 1402c, a primary flexible delivery element 1404, and an extension delivery element 1406. The instrument hubs 1402, for example, may include any style of hub described herein, such as the hub 102 of FIG. 1, the hub 202 of FIG. 2A, the hub 402 of FIG. 4A, and/or the fluid management hub 700 of FIG. 7. Any of the hubs 1402 may be releasably connected to a connecting member 1408 of the proximal end of the flexible delivery element 1404 or a connecting member 1410 of the extension delivery element 1406. Further, the connecting member 1408 of the flexible delivery element 1404 may be releasably connected to a mating member 1412 of the extension delivery element 1406 to create an additional length of delivery element, thereby enabling access to remote treatment sites. Although illustrated as including the retaining member style described in relation to FIG. 12A through FIG. 12D, in other embodiments, the connecting members 1408, 1410 may include different styles of mating components, examples of which have been described above. Further, although illustrated for simplicity's sake as being generally uniform in diameter and shape, in other embodiments, the flexible delivery element 1404 and/or the extension delivery element 1406 may include varying diameters, flexibilities, and/or curved shapes as described in greater detail in relation to FIG. 8 and FIG. 15.

In some embodiments, an instrumentation system such as the instrumentation system 1400 includes multiple flexible delivery elements 1404. For example, an instrumentation kit may include at least one of the instrument hubs 1402 along with two or more flexible delivery elements 1404 of varying lengths and/or diameters. In an illustrative example, an instrumentation kit may include a first flexible delivery element 1404 having an 8 French compatibility and a second flexible delivery element having a six French compatibility. Further to the illustrative example, the instrumentation kit may include the extension delivery element 1406 or multiple extension delivery elements (e.g., one six French compatible and one eight French compatible).

While exchanging removable connectors and/or control elements during a procedure using tools such as those described in relation to the preceding figures, in some embodiments, a medical professional will want to retain an extending element (e.g., guidewire or microcatheter) at a procedural site. However, the movements of the practitioner, the blood flow through the access path through the arterial site, and/or the movements of various equipment (e.g., the sliding off of one catheter and/or overlaying of another catheter, etc.) can cause the extending element to drift from its position. The following figures and description discuss various solutions for retaining the position of an extending element while adjusting the equipment being used during a therapeutic procedure. Certain solutions (security devices or elements), in some implementations, are introduced over the flexible extending element after removal of at least a portion of the connectors and/or control elements.

Turning to FIG. 8, an example flexible shaft 800 includes a curved section 802 for selectably locking a position of a flexible extending element 804 (e.g., guidewire, microcatheter, etc.) within an internal lumen of the flexible shaft 800. The flexible extending element 804, for example, may be frictionally retained in the “S”-style curve of the flexible shaft 800, prohibiting movement of the flexible extending element 804 in at least a lengthwise direction of the flexible shaft 800. In use, an external support element (e.g., microcatheter, hypotube, sleeve, etc.) may be slid over at least the curved section 802 of the flexible shaft 800 to cause the “S”-style curve to straighten, thereby freeing the flexible extending element 804 to move within the flexible shaft 800. When performing operations that may accidentally dislodge a distal position of the flexible extending element 804, a practitioner may slide the external support element off of the curved section 802 to effectively lock the position of the flexible extending element 804. The operations, in some examples, may include exchanging an outer delivery element (e.g., catheter or sleeve), exchanging or adding a proximal connection element (e.g., instrument hub, fluid management system, etc.), or removing an outer delivery element and/or proximal connection element. In an illustrative example, an external support element may cover the curved section 802 of the flexible shaft 800 during use, and the external support element may be at least partially removed from the flexible shaft 800 to allow the curved section 802 to return to its pre-formed curvature. In some embodiments, the “S”-style curve is created in the flexible shaft 800 using a memory shape polymer, metal or metal braid, and/or an alloy such as stainless steel and/or nickel titanium (Nitinol®).

Conversely, in some embodiments (not illustrated), a shape of a flexible shaft is modified using an external shaping element (e.g., an outer sheath or sleeve, etc.) to cause the flexible shaft to curve, thereby frictionally trapping the flexible extending element in a present position. In an illustrative example, the external shaping element may be introduced over a proximal end of the flexible extending element and, thereafter, over the flexible shaft to cause the flexible shaft to form to the curve of the external shaping element. The external shaping element, for example, may be constructed using a braided metal or polymer. The braid can be bare or coated with a soft elastomeric polymer. The external shaping element is formed of a shapable material, in some embodiments, such that it bends or “clicks” into a curved position for locking and manually straightens when adjusted in a reverse direction to enable movement of the extending element.

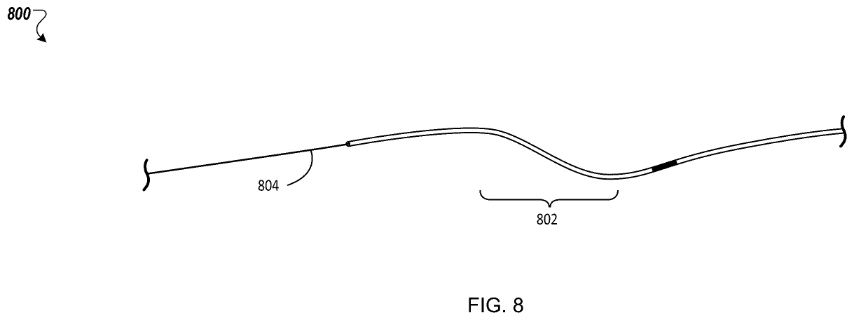

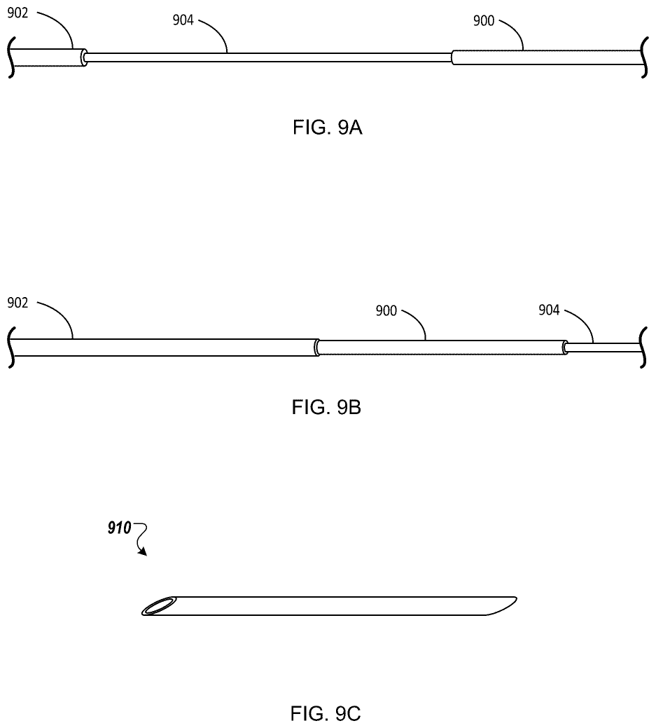

Turning to FIG. 9A and FIG. 9B, in some embodiments, a wedge element 900 is introduced between a flexible shaft 902 and a flexible extending element 904 to fill a gap between the flexible shaft 902 and the flexible extending element 904, thereby creating a friction “wedge” that functions as a locking mechanism to retain a present position of the flexible extending element 904. As illustrated in FIG. 9A, the wedge element 900 is slidably disposed along the flexible extending element 904, external to the flexible shaft element 902. To lock the position of the flexible extending element 904, turning to FIG. 9B, the wedge element 900 may be slid along the flexible extending element 904 to the flexible shaft 902 so that a portion of the wedge element 900 can be inserted into the flexible shaft 902, thereby preventing movement of the flexible extending element 904. The flexible shaft 902, for example, may be added to the flexible extending element 904 after removing any connection elements (e.g., shaft mating element, shaft locking element, hub element, etc.) from the proximal end of the flexible shaft 902.

Although illustrated as having a substantially consistent outer dimension, to aid in alignment and wedging while avoiding pushing the wedge element 900 into the flexible shaft 902 to a point where it may be difficult to grasp to remove, in some embodiments, the wedge element 900 has a tapered outer diameter, such that a smaller diameter end leads in inserting the wedge element 900 into the flexible shaft 902, and the wedge element 900 can be inserted into the flexible shaft 902 to the point at which the diameter of the section of the wedge element 900 abutting the edge of the flexible shaft 902 is so similar in diameter to the inner diameter of the flexible shaft 902 that the wedge element 900 fits no further inside of the flexible shaft 902.

Turning to FIG. 9C, in some embodiments, a wedge element 910 is formed with a bias-cut edge such that a first portion of the wedge element 910 is slid into the flexible shaft 902, gradually followed by an increasing portion of the diameter of the wedge element 910 until a full diameter of a portion of the wedge element 910 is inserted inside of the flexible shaft 902. Rather than needing to carefully align the wedge element 910 with the flexible shaft 902, for example, the practitioner would simply need to insert the leading edge of the wedge element 910, and in sliding the wedge element 910 into the flexible shaft 902, the diameters would come into alignment. This is beneficial where visibility may be less than ideal, and the flexible shaft 902 and wedge element 910 are small and could be difficult to manually maneuver.

The wedge element 900 and/or 910, for example, may be formed as a rigid or semi-rigid tube, straw, or long hollow conical shape with a graduated diameter. In some examples, the wedge element 900 and/or 910 may be constructed using a metal, polymer, and/or metal alloy.

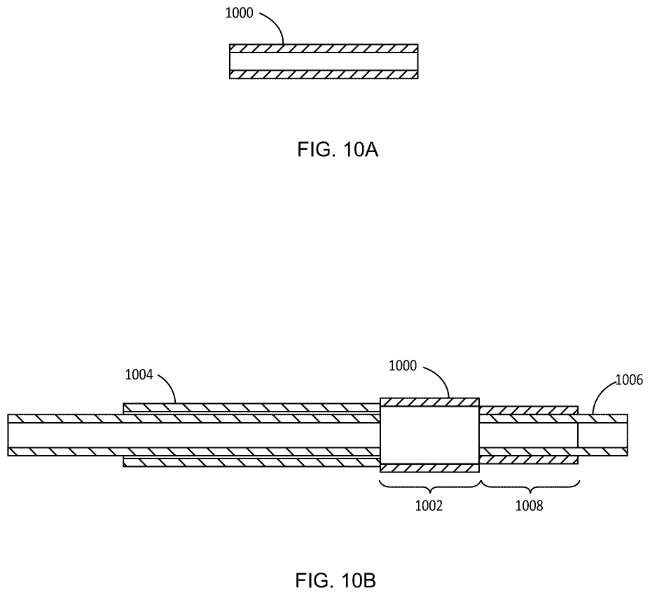

FIG. 10A and FIG. 10B illustrate an example sleeve-type guidewire position locking mechanism (sleeve element) 1000 for use with a flexible shaft. The stretchable (elastic) sleeve element 1000, for example, may be formed of elastic polymers which can be stretched over both a flexible shaft 1004 and a flexible extending element 1006 to hold them in place relative to each other. The sleeve element 1000 may be formed of a soft elastic polymer with shape memory, such as, in some examples, silicone, Tecoflex® (by Microspec Corporation of Peterborough, NH) or other medical-grade aliphatic polyether polyurethane, nylon, Pebax® (by Arkema, Inc. of King of Prussia, Pennsylvania) or other polyether block amide (PEBA), and/or polyurethane. In use, the sleeve element 1000 may be introduced along the proximal end of the flexible extending element 1006, moved to a region 1008 of the flexible extending element 1006 abutting the proximal end of the flexible shaft 1004, and pulled over a proximal region 1002 of the flexible shaft 1004 to compressively retain the relative positions of the flexible shaft 1004 and the flexible extending element 1006.

As illustrated in FIG. 10A, the sleeve element 1000 includes consistent dimensions (e.g., a constant diameter). The material of the sleeve element 1000, in some implementations, is uniform throughout. In other implementations, the material of the sleeve element 1000 may be designed to provide increased compressive force on the flexible extending element 1006 while allowing for greater ease to the medical practitioner in stretching the distal end of the sleeve element over the proximal end of the flexible shaft 1004.

In other embodiments, the sleeve element may include a larger end for capturing the proximal end of the flexible shaft 1004 and a smaller head to compressively retain the flexible extending element 1006. In this manner, a consistent material may be used, exerting sufficient compressive strength to prevent movement of the flexible extending element 1006 while improving the ease of positioning over the proximal end of the flexible shaft 1004 (e.g., in a shaft region 1002).

Conversely, in further embodiments, a diameter of the flexible shaft 1004 may be smaller (e.g., a thickness of the flexible shaft may be reduced) in a proximal region 1002 configured to be covered by the sleeve element 1000, such that, after positioning of the sleeve element 1000 over the proximal region 1002 and extending proximally to an adjacent region 1008 of the flexible extending element 1006 to secure the relative position of the flexible shaft 1004 and the flexible extending element 1006, a diameter of the region 1002 including the diameter of the flexible shaft 1004 layered with the diameter of the sleeve element 1000 may substantially coincide with a diameter of the flexible shaft distal to the region 1002.

FIG. 15A through FIG. 15C illustrate example portions of lumens having varying flexibility achieved by applying a material treatment along a portion of the length of the example lumen. The lumens, in some examples, may be a portion of a delivery element, tubular extension of a removable hub system, or catheter. Each of the lumens of FIG. 15A through FIG. 15C have been treated using a spiral grinding (e.g., “feathering”) or cutting pattern to selectively adjust the flexibility of the material of the segment of the lumen. The spiral grinding or cutting pattern, for example, may increase the flexibility (e.g., decrease a deflective force value) of the segment of the lumen by at least 50%. For example, a first deflection force value of the original lumen (e.g., polymer material catheter) may be within a range from 5.5 N to 6.5 N, and the second deflection force value of the segment treated with spiral grinding or cutting may be within a range from 2.7 N to 3.2 N The spiral grinding or cutting pattern, in some examples, may be applied through laser ablation and/or razor cutting the portions of the lumens.

Turning to FIG. 15A, a lumen 1500 includes a uniform grind or cut pattern along a length 1502. The grind or cut pattern may be applied through at least a portion of a thickness of the lumen 1500. The depth of the grinding or cutting, for example, may depend in part on a material composition of the lumen 1500. For a flexible material such as a polymer, a fairly shallow cut or grind (e.g., one tenth to one half of the depth of material of the lumen 1500) may significantly alter the flexibility of the lumen 1500 while retaining material stability of the lumen. In the example of a metal hypotube, the metal lumen may be spiral cut and coated with a flexible material, such as a polymer coating. The spiral cut, for example, may transform the rigid metal hypotube to a flexible coil, where the flexibility depends on the density of the spiral cut (e.g., a number of spirals per unit length or a distance between each pair 1504a, 1504b of spiral cuts).

Turning to FIG. 15B, rather than a uniform pattern, in some embodiments, a graduated spiral grinding or cutting pattern may be applied along a length 1512 of a lumen 1510. As illustrated, for example, a distance between a first spiral 1514a and a second spiral 1514b is greater than a distance between the second spiral 1514b and a third spiral 1514c, and so on. By applying an increasingly dense spiral cut, the segment of lumen 1510 may be prepared as a transition segment. For example, the segment of lumen 1510 may be used for transitioning from a rigid (e.g., hypotube-based) section to a flexible (e.g., polymer-based) section. The transition segment, in an illustrative example, may be a transitional hypotube segment configured to interface with a flexible delivery element, such as a catheter. In the example of a polymer catheter or other delivery element, the length 1512 of the lumen 1510 may be gradually adjusted in flexibility to introduce a flex region having increased flexibility (e.g., similar to a “flexible straw” where a flexing section allows for curving while an abutting section (e.g., the drinking section) is relatively more rigid). Although the graduated spiral cut is illustrated as increasing in one direction, in other embodiments the variation may be applied in the opposite direction (e.g., wider to narrower from an uncut end 1516). In further embodiments, the graduated spiral cut may both increase and decrease in density (e.g., graduating towards and away from a segment with greatest flexibility).

Turning to FIG. 15C, rather than uniformly cutting or grinding through an entire circumference of each spiral 1524 of the spiral pattern of a length 1522 of a lumen 1520, in some embodiments, the cutting or grinding may be applied in a directional fashion, such that the resultant material is “trained” to flex in a particular direction (e.g., a steerable catheter end). In illustration, the grinding may be performed such that a selected side 1526 (e.g., approximately one sixth to one half of the circumference) of the lumen 1520 is thinned in a consistent or graduated manner along the length 1522 to encourage increased flexibility in a direction 1528 deflecting from the selected side 1526.

While certain embodiments have been described, these embodiments have been presented by way of example only and are not intended to limit the scope of the present disclosures. Indeed, the novel methods, apparatuses and systems described herein can be embodied in a variety of other forms; furthermore, various omissions, substitutions and changes in the form of the methods, apparatuses and systems described herein can be made without departing from the spirit of the present disclosures. The accompanying claims and their equivalents are intended to cover such forms or modifications as would fall within the scope and spirit of the present disclosures.

1. A system for adjusting catheter instrumentation during a vascular interventional procedure, the system comprising:
a flexible shaft comprising at least one lumen, a first lumen of the at least one lumen configured for passing a flexible extending element therethrough;
a removable instrument hub configured to releasably couple to a proximal end of the flexible shaft, the removable instrument hub comprising
a hub element comprising at least one grasping feature for securely retaining the hub element in a hand of an operator, the hub element comprising at least one opening for passing the flexible extending element therethrough, and
a tubular member extending from a distal end of the hub element; and
at least one mating feature arranged on one of i) the proximal end of the flexible shaft or ii) a distal end of the tubular member, the at least one mating feature being configured to frictionally mate with an interior surface of the other of i) the proximal end of the flexible shaft or ii) the distal end of the tubular member, such that, upon insertion of the at least one mating feature into i) the proximal end of the flexible shaft or ii) the distal end of the tubular member,
the removable instrument hub is releasably coupled to the flexible shaft, and
the tubular member is in fluid communication with the flexible shaft.

2. The system of claim 1, wherein:
the tubular member has a distal diameter larger than a proximal diameter of the flexible shaft; and
the at least one mating feature is arranged on an exterior surface of the flexible shaft.

3. The system of claim 2, wherein the at least one mating feature and at least a proximal section of the flexible shaft are formed of a same material.

4. The system of claim 3, wherein:
the proximal section of the flexible shaft is composed of a rigid or semi-rigid material; and
a polymer-based section of the flexible shaft is overlappingly attached to the proximal section of the flexible shaft.

5. The system of claim 2, wherein the at least one mating feature comprises a plurality of barbs or a plurality of wedge-shaped protrusions aligned substantially parallel to a length of the tubular member.

6. The system of claim 2, wherein an interior diameter of the tubular member widens in a distal direction to accept and guide the at least one mating feature of the flexible shaft into a mating position.

7. The system of claim 1, wherein:
the tubular member has a distal diameter smaller than a proximal diameter of the flexible shaft; and
the at least one mating feature is arranged on an exterior surface of the tubular member.

8. The system of claim 7, wherein the at least one mating feature and at least a distal section of the tubular member are formed of a same material.

9. The system of claim 7, wherein the at least one mating feature comprises a plurality of barbs or a plurality of wedge-shaped protrusions aligned substantially parallel to a length of the tubular member.

10. The system of claim 1, wherein the removable instrument hub is a fluid management instrument hub.

11. The system of claim 10, further comprising a second removable instrument hub different than the removable instrument hub, wherein:
the second removable instrument hub comprises a second tubular member; and
the second removable instrument hub is configured to releasably couple to the flexible shaft using at least one of i) the at least one mating feature or ii) at least one second mating feature arranged at the distal end of the second tubular member.

12. The system of claim 1, wherein the at least one mating feature is formed of a flexible material configured to compress when inserted into one of a) the first lumen of the flexible shaft or b) the tubular member.

13. The system of claim 12, wherein the flexible material comprises one or more polymers.

14. The system of claim 1, wherein the tubular member is formed of a rigid material.

15. The system of claim 14, wherein the tubular member comprises a hypotube.

16. The system of claim 1, wherein the at least one mating feature comprises a plurality of ridged members.

17. The system of claim 1, wherein:
the at least one mating feature is arranged at a proximal end of the flexible shaft; and
at least one coordinating mating feature is arranged at a distal end of the tubular member.

18. The system of claim 17, wherein a first of the at least one mating feature or the at least one coordinating mating feature comprises a ridge arranged around a majority of an interior diameter and another of the at least one mating feature of the at least one coordinating mating feature comprises a plurality of wedge-shaped members configured to push beyond and be held by the ridge.

19. The system of claim 1, further comprising an extension shaft, the extension shaft comprising:
one or more lumens, a guide lumen of the one or more lumens configured for passing the flexible extending element therethrough; and
one or more retention features arranged on a distal end of the extension shaft, the one or more retention features configured to releasably connect with the at least one mating feature of the proximal end of the flexible shaft to effectively increase a length of the flexible shaft.

20. The system of claim 1, further comprising a second flexible shaft comprising one or more lumens, wherein:
the second flexible shaft has a second outer diameter compatible with a different standard instrumentation size than a first outer diameter of the flexible shaft; and
the removable instrument hub is configured to releasably couple to a proximal end of the second flexible shaft.

21.-59. (canceled)
